SEQUENCE LISTING (2 sequences)

SEQ ID NO: 1 - protein, 21 aa, Homo sapiens
GIVEQCCTSICSLYQLENYCG

SEQ ID NO: 2 - protein, 32 aa, Homo sapiens
FVNQHLCGSHLVEALYLVCGERGFFYTPKTRR

(19) United States
(12) Patent Application Publication
(10) Pub. No.: US 2019/0175737 A1
(43) Pub. Date: Jun. 13, 2019

(54) INSULIN GLARGINE AND TRIETHYLENETETRAMINE (TETA)

(71) Applicant: Arecor Limited, Saffron Walden, Cambridgeshire (GB)

(72) Inventors: Jan JEZEK, Saffron Walden, Cambridgeshire (GB); Sarah HOWELL, Saffron Walden, Cambridgeshire (GB); David GERRING, Saffron Walden, Cambridgeshire (GB); Gary WATTS, Saffron Walden, Cambridgeshire (GB); Leon ZAKRZEWSKI, Saffron Walden, Cambridgeshire (GB)

(21) Appl. No.: 16/324,726

(22) PCT Filed: Aug. 11, 2017
(86) PCT No.: PCT/GB2017/052378

§ 371 Date: Feb. 11, 2019

(30) Foreign Application Priority Data
Aug. 12, 2016 (GB) 1613896.8

(51) Int. Cl. A61K 47/18; A61K 38/28; A61K 9/08; A61K 47/10; A61K 47/26; A61K 47/14; A61K 9/00

(52) U.S. Cl. CPC A61K 47/18; A61K 38/28; A61K 9/08; A61K 9/0019; A61K 47/26; A61K 47/14; A61K 47/10

(57) ABSTRACT

There is provided inter alia an aqueous solution composition comprising insulin glargine as an active ingredient and triethylenetetramine (TETA) as a stabilising agent.

This invention relates to aqueous solution compositions of insulin glargine and their use in therapy, particularly in applications where chronic administration of insulin glargine is desirable.

### BACKGROUND

The current insulin market comprises a number of different products, including regular human insulin (RHI) and a number of insulin analogues. The analogues have been developed by amino acid substitutions to provide certain advantages over RHI. In particular, the advantages relate to the speed of action of insulin. Some analogues (e.g. insulin lispro, insulin aspart and insulin glulisine) have been developed to provide a faster onset of action and are therefore the preferred option for prandial use (i.e. before/with meal). These are referred to as ‘rapid acting insulin analogues’. In contrast, other analogues such as insulin glargine and insulin degludec have been developed to act very slowly and are thus used as basal insulins providing basal glycaemic control for ˜24 hours period. These are referred to as ‘long acting insulin analogues’ or ‘slow acting insulin analogues’ or ‘basal insulins’.

Insulin glargine is a basal insulin analogue resulting from the substitution of a glycine residue at position 21 of the A-chain of an insulin molecule for asparagine and the addition of two arginine residues to the B-chain at position 30 (and is sometimes represented as Gly(A21),Arg(B31),Arg(B32)-human insulin). These structural modifications cause a shift in the isoelectric point of the molecule, rendering it more soluble at an acidic pH and significantly decreasing its solubility at physiological pH. Insulin glargine is typically formulated at pH 4 in the absence of a buffer. Following injection into the (pH-neutral) subcutaneous tissue, insulin glargine forms microprecipitates from which it is subsequently slowly released (http://www.lantus.com/hcp/about-lantus/how-lantus-works). The precipitation is a direct consequence of pH change from 4 to ˜7.4 (physiological pH). This slow release ensures that small amounts of insulin glargine are released into the body continuously, giving an almost peakless profile.

Insulin glargine has been marketed as a 100 U/ml product by Sanofi Aventis under the brand name Lantus®. More recently, Sanofi Aventis started marketing a 300 U/ml insulin glargine product under the brand name Toujeo®, which was shown to have a more prolonged PK/PD profile compared with Lantus®, resulting in longer glucose control. In addition, a biosimilar version of insulin glargine (100 U/ml) has been launched by Eli Lilly under the brand name Abasaglar®.

Lantus® is marketed both in a pen presentation and in a multi-dose vial presentation. The composition of Lantus® for a pen cartridge is as follows: 100 U/ml insulin glargine (equivalent to approximately 3.6 mg/ml); 25 mM m-cresol (a preservative); 185 mM glycerol and 30 μg/ml ionic zinc (from zinc oxide or zinc chloride); at pH 4.0.

The composition of Lantus® for vials is identical to that for the pen cartridge, except that it also contains polysorbate 20 (20 μg/ml), which is added to the vial formulation in order to prevent agitation-related aggregation. Agitation-related aggregation is a significant concern for the vial presentation due to the presence of a headspace, but not for the pen cartridge where there is no headspace.

Abasaglar® is currently only available in a pen presentation and the composition is identical to that of Lantus®.

Toujeo® (containing 300 U/ml insulin glargine) is formulated together with the same components at the same concentrations as for Lantus® with the exception of the ionic zinc concentration—the insulin/zinc (w/w) ratio maintained is identical to that of the Lantus® product, so the zinc level is 90 μg/ml in Toujeo®.

WO2013/114112 (ARECOR LIMITED) discloses an aqueous solution comprising an antibody protein at a concentration of at least about 10 mg/mL and an oligomer of ethyleneimine, wherein the number of repeating units of ethyleneimine (n) in the oligomer is in the range n=2-12.

Torosantucci et al. (European Journal of Pharmaceutics and Biopharmaceutics, 2013, 84, pp 464-471) describes investigations into the ability of, inter alia, triethylenetetramine, to prevent protein oxidation, aggregation and fragmentation observed in the presence of the oxidative system of Cu²⁺/ascorbate.

The currently marketed insulin glargine products must be stored at 2-8° C. prior to use. During the 28 days in-use period (i.e. period starting from the first use of the pen cartridge or vial) the product can be kept at temperatures up to 30° C. (refrigerated or unrefrigerated at 15-30° C. is recommended).

In order to improve patients' convenience and shipment logistics there is a need for insulin glargine compositions with improved stability, in particular compositions wherein:
the temperature at which the product can be kept during the in-use period is increased, e.g. to 40° C.;
the duration of the in-use period is increased, e.g. to 2 months, preferably 3 months;
the product can be stored at increased temperature, such as controlled room temperature (20-25° C.) for part of the shelf-life, e.g. 3 months, 6 months or the entire shelf-life, whilst maintaining the in-use stability at 30° C. for 28 days.

Thus, an object of the present invention is the provision of an aqueous solution composition of insulin glargine as active ingredient with improved stability.

### SUMMARY OF THE INVENTION

Thus, according to the invention, there is provided an aqueous solution composition comprising insulin glargine as an active ingredient and triethylenetetramine (TETA) as a stabilising agent.

### FIGURES

FIG. 1: Stability of insulin glargine (100 U/ml) in the presence of sodium chloride assessed by SEC following storage at 30° C. for 4 and 12 weeks (see Table 2 of Example 1).

FIG. 2: Stability of insulin glargine (100 U/ml) in the presence of sodium chloride assessed by RP-HPLC following storage at 30° C. for 4 and 12 weeks (see Table 3 of Example 1).

FIG. 3: Stability of insulin glargine (100 U/ml) in the presence of TETA assessed by SEC following storage at 30° C. for 4 and 12 weeks (see Table 5 of Example 2).

FIG. 4: Stability of insulin glargine (100 U/ml) in the presence of TETA assessed by RP-HPLC following storage at 30° C. for 4 and 12 weeks (see Table 6 of Example 2).

FIG. 5: Stability of insulin glargine (100 U/ml) in the presence of TETA assessed by SEC following storage at 30° C. for 4 and 8 weeks (see Table 8 of Example 3).

FIG. 6: Stability of insulin glargine (100 U/ml) in the presence of TETA assessed by RP-HPLC following storage at 30° C. for 4 and 8 weeks (see Table 9 of Example 3).

FIG. 7: Stability of insulin glargine (100 U/ml) in the presence of TETA assessed by SEC following storage at 30° C. for 4 and 8 weeks (see Table 11 of Example 4).

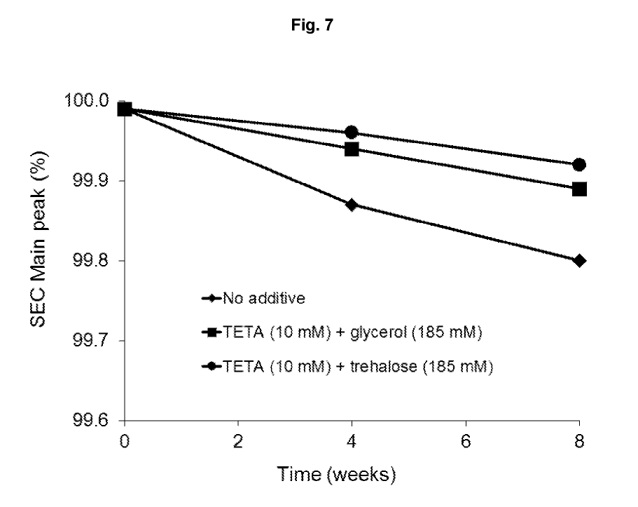

FIG. 8: Stability of insulin glargine (100 U/ml) in the presence of TETA assessed by RP-HPLC following storage at 30° C. for 4 and 8 weeks (see Table 12 of Example 4).

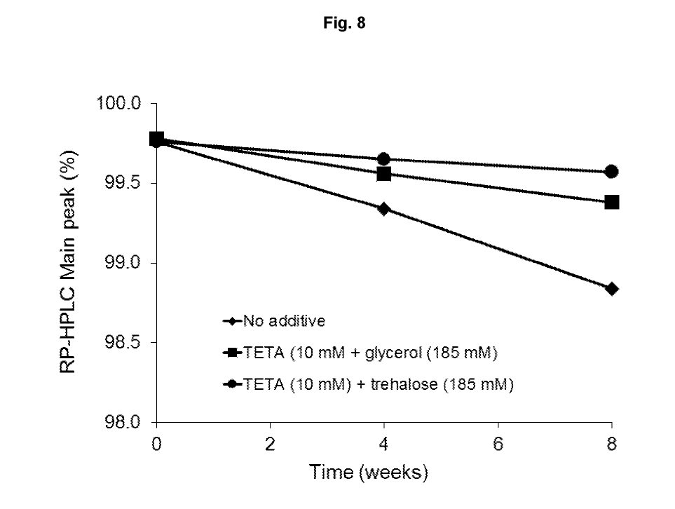

### DESCRIPTION OF THE SEQUENCE LISTING

SEQ ID NO. 1: A-chain of insulin glargine

SEQ ID NO. 2: B-chain of insulin glargine

### DETAILED DESCRIPTION OF THE INVENTION

As used herein “insulin glargine” refers to the analogue of native human insulin having an A-chain as set out in SEQ ID NO. 1 and a B-chain as set out in SEQ ID NO. 2, and containing and connected by disulfide bridges as in the native molecule (Cys A6-Cys A 11, Cys B7 to Cys A7 and Cys-B19-Cys A20). Reference to a “composition” herein is intended to refer to an aqueous solution composition.

The concentration of insulin glargine in the composition is typically between 10 U/ml and 1000 U/ml, for example between 50 U/ml and 500 U/ml, or between 100 U/ml and 200 U/ml. Alternatively the concentration of insulin glargine in the composition may be between 200 U/ml and 500 U/ml or between 500 U/ml and 700 U/ml. In one embodiment, the concentration of insulin glargine is about 100 U/ml. In one embodiment, the concentration of insulin glargine is about 200 U/ml. In another embodiment, the concentration of insulin glargine is about 300 U/ml. In another embodiment, the concentration of insulin glargine is about 400 U/ml. In another embodiment, the concentration of insulin glargine is about 500 U/ml. “U/ml” as used herein describes the concentration of insulin glargine in terms of a unit per volume, wherein “U” describes the activity of insulin glargine that is biologically equivalent to 34.7 μg of pure crystalline insulin. 100 U corresponds to 3.64 mg of insulin glargine.

The composition of the invention includes triethylenetetramine (TETA) as a stabilising agent. The TETA is suitably present at a concentration of 1-50 mM, for example 2-45 mM, 5-45 mM, 2-40 mM, 5-40 mM or 2-25 mM such as 5-15 mM, 6-14 mM, 7-13 mM, 8-12 mM.

The pH of the composition is suitably between 3 and 5, for example between 3.5 and 4.5. Preferably it is around pH 4. Insulin glargine in a composition at such a pH is typically completely soluble.

The composition may additionally comprise a tonicity modifier, which may be charged or uncharged. Examples of uncharged tonicity modifiers include glycerol, 1,2-propanediol, mannitol, sorbitol, trehalose, PEG300 and PEG400. When included, an uncharged tonicity modifier is typically added at a concentration of 50-1000 mM, for example 100-500 mM, such as about 300 mM. Examples of charged tonicity modifiers include sodium chloride, sodium sulfate, and amino acids such as glycine or arginine. When included, a charged tonicity modifier is typically added at a concentration of 25-500 mM, for example 50-250 mM such as about 150 mM. An uncharged tonicity modifier rather than a charged tonicity modifier is generally preferred.

The composition may additionally comprise a surfactant. In one embodiment, the surfactant is a non-ionic surfactant. In another embodiment, the surfactant is a cationic surfactant. Suitable cationic surfactants include benzalkonium and benzethonium salts. In one embodiment, the cationic surfactant is selected from benzethonium salts e.g. benzethonium halide such as benzethonium chloride. In another embodiment, the cationic surfactant is selected from benzalkonium salts e.g. benzalkonium halide such as benzalkonium chloride. In a further embodiment, the cationic surfactant is a mixture of benzethonium salts and benzalkonium salts such as a mixture of benzethonium chloride and benzalkonium chloride.

When included, the surfactant is typically at a concentration of 5-200 μM, such as 10-100 μM or 20-100 μM.

The compositions of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol.

The concentration of preservative is typically 1-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

The composition may comprise a buffer. Suitable buffers include acetate, succinate, citrate, histidine, malate and maleate.

The composition suitably comprises ionic zinc i.e. Zn²⁺ cations. The source of ionic zinc will typically be a water soluble zinc salt such as ZnCl₂, ZnO, ZnSO₄, Zn(NO₃)₂ or Zn(acetate)₂ and is most suitably ZnCl₂ or ZnO. Suitably, the amount of ionic zinc present is between 10-100 μg per 100 U of insulin glargine, such as 20-50 μg, e.g. around 30 μg per 100 U of insulin glargine. The amount of ionic zinc in the concentration calculation does not include the mass of the counter ion.

Suitably the overall concentration of charged species in the composition is low. In the context of this invention, a charged species is defined as a chemical entity which carries at least one charge under the conditions of the composition, e.g. sodium cation (Na⁺), chloride anion (Cl⁻) or an amino acid such as histidine. Suitably, the overall concentration of charged species, other than those originating from ionic zinc, TETA and insulin glargine in the composition is less than 150 mM, for example less than 100 mM, such as less than 50 mM or less than 25 mM. In one embodiment the composition is substantially free of ionic species (apart from insulin glargine, ionic zinc and TETA) which possess more than one charged group or which have a total charge of more than 1. For example, the composition is substantially free of ionic species (apart from insulin glargine, ionic zinc and TETA) which possess 2, 3, 4 or more charged groups. For example, the composition is substantially free of ionic species (apart from insulin glargine, ionic zinc and TETA) which have a total charge of 2, 3, 4 or more.

The ionic strength of a composition may be calculated according to the formula:

$$I = 0.5 \times {\sum }_{X = 1}^{n} {c}_{x} {z}_{x}^{2}$$

in which cₓ, is molar concentration of ion x (mol L⁻¹), zₓ, is the absolute value of the charge of ion x and the sum covers all ions (n) present in the composition. The contribution of insulin glargine itself and of the ionic zinc, and of TETA should be ignored for the purposes of the calculation. The ionic strength of the composition is suitably kept to a minimum level since higher ionic strength compositions are typically less stable than comparable lower ionic strength compositions. Suitably the total ionic strength of the composition is less than 40 mM, e.g. less than 20 mM, e.g. less than 10 mM.

A specific embodiment that may be envisaged is a composition according to the invention with a pH of about 4, comprising insulin glargine as an active ingredient at a concentration of between 50 U/mL and 500 U/mL, TETA as a stabilising agent at a concentration of 2-45 mM e.g. 5-40 mM, and an uncharged tonicity modifier selected from glycerol, 1,2-propanediol, mannitol, sorbitol, trehalose, PEG300 and PEG400.

The presently claimed invention derives from the surprising observation that compositions of insulin glargine as an active ingredient are stabilized by the addition of triethylenetetramine (TETA). An improvement in both chemical and physical stability is observed (see Examples 2-4).

This result was surprising in view of the inventors' observation that increasing the ionic strength of insulin glargine composition results in a decrease in physical and chemical stability (see Example 1). At the acidic pH typically used to formulate insulin glargine, TETA is charged, and would therefore increase the ionic strength of the composition. Based on the observations of Example 1, it would be expected that an increase in ionic strength would result in no change or a decrease in stability.

As can be seen from Examples 2 and 3, the addition of TETA produced a notable enhancement in stability. Without wishing to be bound by theory, it appears that the claimed composition provides optimum components and concentrations for the stability of insulin glargine, resulting from a balance between a stabilizing effect of TETA, and the destabilizing effect of increasing the ionic strength of the composition.

As shown in Examples 2 and 3, compositions of the invention demonstrated good stability following storage at 30° C. for 4 weeks, 8 weeks and 12 weeks.

Enhanced stability was also observed for higher concentrations of insulin glargine (500 U/ml), as shown in Example 4.

The physical stability of an insulin glargine composition refers to the tendency of the insulin glargine molecule to form insoluble aggregates due to, inter alia, destabilizing interactions with surfaces and interfaces, and temperature fluctuations. Aggregates are described herein as “high molecular weight species”, which refers to any irreversibly formed component of protein content which has an apparent molecular weight at least double the molecular weight of the parent insulin glargine molecule. Thus, high molecular weight species are multimeric aggregates of the parent insulin glargine molecule, which may comprise the parent insulin glargine molecules with considerably altered conformation or they may be an assembly of the parent insulin glargine units in the native or near-native conformation. Physical stability of the insulin glargine composition can be evaluated by methods known in the art, including by visual inspection, size exclusion chromatography (SEC), electrophoresis, analytical ultracentrifugation, light scattering, dynamic light scattering, static light scattering and field flow fractionation. Exemplary methods using visual inspection and size exclusion chromatography (SEC) are described in the General Procedures.

The chemical stability of an insulin glargine composition refers to changes in the covalent protein structure of the insulin glargine molecule leading to the formation of chemically related insulin glargine species (degradation products). Chemical stability of the insulin glargine composition can be evaluated by reversed phase HPLC (RP-HPLC) to determine the proportion of total chemically related insulin glargine species (i.e. species generated during storage or other stress conditions by chemical modification of insulin glargine, including, deamidation of glutamine or asparagine residues, cyclic imide formation or various hydrolysis processes), as described in the General Procedures.

Suitably the composition of the invention remains as a clear solution for a longer period of time compared to the current commercially available insulin glargine products, allowing for a longer in-use period. Thus, in one embodiment, the composition of the invention remains as a clear solution during storage at 15-30° C. e.g. at 30° C. for longer than 4 weeks, for example for 5 weeks, 6 weeks, 7 weeks, 8 weeks, 10 weeks or 12 weeks. By “clear solution” it is meant that no visible precipitation is observed during storage.

Suitably the composition of the invention remains as a clear solution when exposed to temperatures which are higher than those recommended for the current commercially available insulin glargine products. Thus, in one embodiment, the composition of the invention remains as a clear solution during storage for 4 weeks, at temperatures greater than 30° C., for example during storage for 4 weeks at 32° C., at 34° C., at 35° C., at 37° C., at 38° C. or at 40° C.

Preferably, the composition of the invention provides a more user-friendly in-use period in that the composition can be stored both at higher temperatures, and for longer periods of time, than recommended for the current commercially available insulin glargine products. Thus, in one embodiment, the composition of the invention remains as a clear solution during storage at 32° C. or higher, for example at 32° C., 34° C., at 35° C., at 37° C., at 38° C. or at 40° C.; for at least 4 weeks, for example for 4 weeks, 5 weeks, 6 weeks, 7 weeks, 8 weeks, 10 weeks or 12 weeks.

In one embodiment, the composition according to the invention is a clear solution with low viscosity (e.g. dynamic viscosity of less than 20 cP, such as less than 10 cP, e.g. less than 5 cP at 25° C. measured using a microfluidics capillary extrusion viscometer, such as m-VROC ™, RheoSense Inc.).

Suitably the composition of the invention has improved storage stability at increased temperature, while maintaining the in-use stability. Thus, in one embodiment, the composition of the invention remains as a clear solution during storage at 25° C. for at least 3 months, for example 3 months, 6 months, 12 months or 24 months; and also remains as a clear solution during an in-use period of 28 days at 30° C., starting immediately after the end of the storage period.

In one embodiment, the composition of the invention comprises no more than 4% (by total weight of insulin glargine in the composition) of high molecular weight species, preferably no more than 3%, 2%, or 1%, following storage at 15-30° C. e.g. 30° C., for longer than 4 weeks, for example 5 weeks, 6 weeks, 7 weeks, 8 weeks, 10 weeks or 12 weeks. By “clear solution” it is meant that no visible precipitation is observed during storage.

In one embodiment, the composition of the invention comprises no more than 4% (by total weight of insulin glargine in the composition) of high molecular weight species, preferably no more than 3%, 2% or 1%, following storage for at least 4 weeks, at temperatures greater than 30° C., for example during storage for 4 weeks at 32° C., 34° C., 35° C., 37° C., 38° C. or 40° C.

In one embodiment, the composition of the invention comprises no more than 4% (by total weight of insulin glargine in the composition) of high molecular weight species, preferably no more than 3%, 2% or 1%, following storage at 32° C. or higher, for example 32° C., 34° C., 35° C., 37 ° C., 38° C. or 40° C.; for at least 4 weeks, for example for 4 weeks, 5 weeks, 6 weeks, 7 weeks, 8 weeks, 10 weeks or 12 weeks.

In one embodiment, the composition of the invention comprises no more than 4% (by total weight of insulin glargine in the composition) of high molecular weight species, preferably no more than 3%, 2%, or 1% following storage at 25° C. for at least 3 months, such as 3 months, 6 months, 12 months or 24 months; and also comprises no more than 4% (by total weight of insulin glargine in the composition) of high molecular weight species, preferably no more than 3%, 2% or 1% following an in-use period of 28 days at 30° C., starting immediately after the end of the storage period.

In one embodiment, a composition of the present invention exhibits an increase in high molecular weight species during storage which is at least 10% lower, preferably at least 25% lower, more preferably at least 50% lower, than a composition lacking TETA as stabilising agent, but otherwise identical, following storage under the same conditions and length of time.

Suitably, a composition of the invention retains at least 95%, e.g. at least 96%, at least 97%, at least 98% or at least 99% native insulin glargine (by total weight of insulin glargine in the composition at time T=0) following storage at 15-30° C. e.g. 30° C., for at least 4 weeks, for example for 4 weeks, 5 weeks, 6 weeks, 7 weeks, 8 weeks, 10 weeks or 12 weeks.

Suitably, a composition of the invention retains at least 95%, e.g. at least 96%, at least 97%, at least 98% or at least 99% native insulin glargine (by total weight of insulin glargine in the composition at time T=0) following storage for at least 4 weeks, at temperatures greater than 30° C., for example during storage for 4 weeks at 32° C., 34° C., 35° C., 37° C., 38° C. or 40° C.

Suitably, a composition of the invention retains at least 95%, e.g. at least 96%, at least 97%, at least 98% or at least 99% native insulin glargine (by total weight of insulin glargine in the composition at time T=0) following storage at 32° C. or higher, for example at 32° C., 34° C., 35° C., 37° C., 38° C. or 40° C., for at least 4 weeks, for example for 4 weeks, 5 weeks, 6 weeks, 7 weeks, 8 weeks, 10 weeks or 12 weeks.

Suitably, a composition of the invention retains at least 95%, e.g. at least 96%, at least 97%, at least 98% or at least 99% native insulin glargine (by total weight of insulin glargine in the composition at time T=0) following storage at 25° C. for at least 3 months, such as 3 months, 6 months, 12 months or 24 months; and also retains at least 95%, e.g. at least 96%, at least 97%, at least 98% or at least 99% native insulin glargine (by total weight of insulin glargine in the composition at time T=0) following an in-use period of 28 days at 30° C., starting immediately after the end of the storage period.

Suitably, a composition of the present invention should exhibit an increase in chemically related insulin glargine species during storage which is at least 10% lower, preferably at least 25% lower, more preferably at least 50% lower, than a composition lacking TETA as a stabilising agent, but otherwise identical, following storage under the same conditions and length of time.

Suitably, the proportion of total chemically related insulin glargine species remains below 4% (by weight), preferably below 3%, more preferably below 2%, and even more preferably below 1% during storage at 15-30° C. e.g. 30° C., for at least 4 weeks, for example 4 weeks, 5 weeks, 6 weeks, 7 weeks, 8 weeks, 10 weeks or 12 weeks.

Suitably, the proportion of total chemically related glargine species remains below 4% (by weight), preferably below 3%, more preferably below 2%, and even more preferably below 1%, during storage for at least 4 weeks, at temperatures greater than 30° C., for example during storage for 4 weeks at 32° C., 34° C., 35° C., 37° C., 38° C. or 40° C.

Suitably, the proportion of total chemically related insulin glargine species remains below 4% (by weight), preferably below 3%, more preferably below 2%, and even more preferably below 1% during storage at least 32° C. or higher, for example at 32° C., 34° C., 35° C., 37° C., 38° C. or 40° C., for at least 4 weeks, for example for 4 weeks, 5 weeks, 6 weeks, 7 weeks, 8 weeks, 10 weeks or 12 weeks.

Suitably, the proportion of total chemically related insulin glargine species remains below 4% (by weight), preferably below 3%, more preferably below 2%, and even more preferably below 1% following storage at 25° C. for 3 months, 6 months, 12 months or 24 months; and also remains below 3% (by weight), preferably below 2%, more preferably below 1% following an in-use period of 28 days at 30° C., starting immediately after the end of the storage period.

In another aspect of the invention, there is provided the use of TETA, for stabilizing an insulin glargine composition.

In a further aspect of the invention, there is provided a method of improving the stability of an aqueous solution composition comprising insulin glargine as an active ingredient which comprises adding TETA to the composition as a stabilising agent.

The composition of the invention is a therapeutic composition.

Thus, in a further aspect of the invention is provided a method of treatment of diabetes mellitus which comprises administering to a subject in need thereof a therapeutically effective amount of a composition as described herein. There is also provided a composition as described herein for use as a pharmaceutical, especially for use in the treatment of diabetes mellitus. For example, said treatment of diabetes mellitus is chronic treatment.

A typical starting dose of a composition of the invention in patients with type 1 diabetes should be approximately one-third of the total daily insulin requirement. Depending on the size and condition this corresponds to 5-50 U, for example 5-25 U such as 15 U, typically administered once a day.

A typical starting dose of a composition of the invention in patients with type 2 diabetes is 0.2 U per kg of body weight, administered once daily, which should subsequently be adjusted to the patient's needs.

There is also provided a container, for example made of plastics or glass, containing one dose or a plurality of doses of the composition as described herein. The container can be for example, a vial, or a cartridge designed to be a replaceable item for use with an injection device.

The compositions of the invention may suitably be packaged for injection, especially sub-cutaneous or intramuscular injection. Sub-cutaneous injection is preferred. Injection may be by conventional syringe or more preferably via a pen device adapted for use by diabetic subjects. Exemplary pen devices include OptiClick®, SoloSTAR® and KwikPen®.

An aspect of the invention is an injection device, particularly a device adapted for subcutaneous or intramuscular injection, for single or multiple use comprising a container containing one dose or a plurality of doses of the composition of the invention together with an injection needle. In an embodiment, the container is a replaceable cartridge which contains a plurality of doses. In an embodiment, the needle is replaceable e.g. after each occasion of use. In one embodiment, the injection device is in the form of a pen.

Another aspect of the invention is a medical device comprising a reservoir comprising a plurality of doses of the composition of the invention and a pump adapted for automatic or remote operation such that upon automatic or remote operation one or more doses of the composition of the invention is administered to the body e.g. subcutaneously or intramuscularly. Such devices may be worn on the outside of the body or implanted in the body.

Insulin glargine-containing compositions according to the invention are expected to have one or more of the advantages of:
good stability during the in-use period at temperatures up to 40° C.;
good stability during an extended in-use period e.g. up to 12 weeks;
good storage stability at an increased temperature e.g. 20-25° C. whilst retaining good in-use stability;

Compositions according to the invention are expected to have good physical and chemical stability as described herein.

### EXAMPLES

### Materials

Insulin glargine used in Examples 1-3 was obtained from HEC Pharma. Insulin glargine used in Example 4 was obtained from a different supplier.

### General procedures

### Analysis of insulin glargine physical stability

### Visual assessment

Visible particles are suitably detected using the 2.9.20. European Pharmacepoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

### Size Exclusion Chromatography (SEC)

Ultra-high performance size exclusion chromatography of insulin glargine preparations is performed using the Waters ACQUITY H-class Bio UPLC® system with a 1.7 μm Ethylene Bridged Hybrid 125 Å pore packing material in a 300 mm by 4.6 mm column. The column is equilibrated in 0.65 mg/ml L-arginine, 20% v/v acetonitrile, 15% v/v glacial acetic acid mobile phase. Flow rate is 0.4 mL/min and UV detection (276 nm) is used. Injection volume is 10 μL. All analyses are performed at ambient temperature.

### Analysis of Insulin Glargine Chemical Stability

### Reversed Phase High-Performance Liquid Chromatography (RP-HPLC)

Ultra-high performance reverse phase chromatography was performed using the Waters ACQUITY H-class Bio UPLC® system with a 1.7 μm Ethylene Bridged Hybrid particle, 130 Å pore resin trifunctionally immobilised with a C18 ligand in a 50 mm by 2.1 mm column. Insulin samples were bound in an 82% w/v Na₂SO₄, 18% v/v acetonitrile, pH 2.3 mobile phase and eluted in 50% w/v Na₂SO₄, 50% v/v acetonitrile gradient flow. 2 μl of sample was acidified with 0.01M HCI and analysed at 0.61 mL/min, with 214 nm UV detection. All analyses were performed at 40° C.

### Example 1—Effect of Ionic Strength (From Inorganic Salts) On Stability of Insulin Glargine (100 U/ml)

The effect of ionic strength on the stability of insulin glargine (100 U/ml) was studied by comparing the stability of insulin glargine (100 U/ml) in the currently marketed formulation of Lantus® both in the absence and in the presence of specific concentrations of sodium chloride and sodium sulphate. The currently marketed formulation of Lantus® contains 25 mM m-cresol, 185 mM glycerol and 30 μg/ml ionic zinc and is adjusted to pH 4.0.

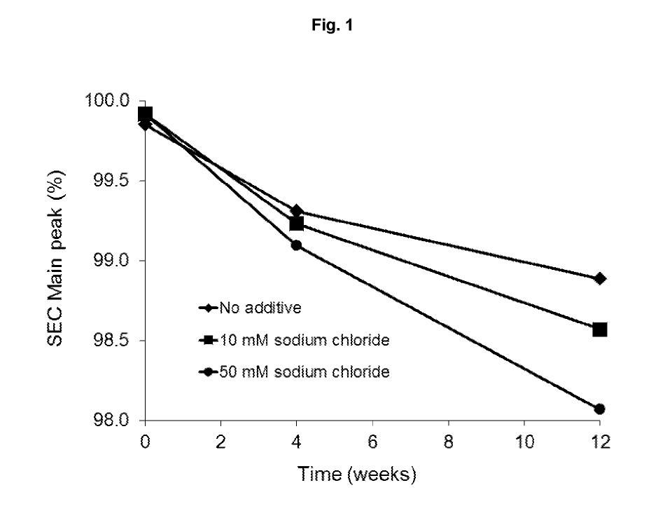

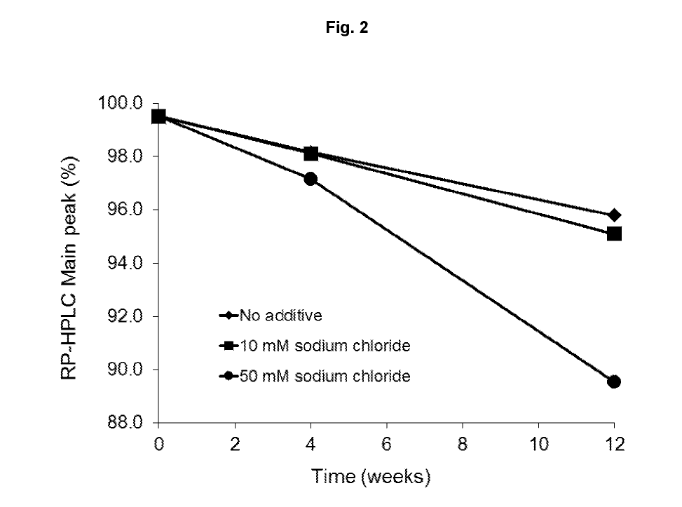

It was shown (Tables 1-3, FIGS. 1-2) that the addition of sodium chloride resulted in impairment of both physical stability (assessed by visual assessment and SEC (see General Procedures)) and chemical stability (assessed by RP-HPLC (see General Procedures)). Sodium sulphate was shown to have a marked impact on physical stability, resulting in considerable precipitation, which prevented further sample analysis by SEC and RP-HPLC.

TABLE 1
Stability of insulin glargine (100 U/ml) assessed by visual assessment
following storage at 30° C. for 4 and 12 weeks. All formulations
contained 25 mM m-cresol, 185 mM glycerol and 30 μg/ml ionic zinc
and were adjusted to pH 4.0. Extent of visible precipitation is graded
on a scale 1-3; 1 = clear solution free of visible particles; 2 = slight
particle formation, 3 = more significant precipitation.

| | Visual | Visual | Visual |
|---|---|---|---|
| | assessment | assessment | assessment |
| Additive | (0 weeks) | (4 weeks) | (12 weeks) |
| None | 1 | 1 | 2 |
| Sodium chloride (10 mM) | 1 | 2 | 2 |
| Sodium chloride (50 mM) | 1 | 3 | 3 |
| Sodium sulphate (10 mM) | 1 | 3 | 3 |
| Sodium sulphate (50 mM) | 2 | 3 | 3 |

TABLE 2
Stability of insulin glargine (100 U/ml) assessed by SEC following
storage at 30° C. for 4 and 12 weeks. All formulations contained
25 mM m-cresol, 185 mM glycerol and 30 μg/ml ionic zinc
and were adjusted to pH 4.0.

| | SEC | | |
|---|---|---|---|
| | main peak | SEC main peak | SEC main peak |
| Additive | (0 weeks) | (4 weeks) | (12 weeks) |
| None | 99.85% | 99.31% | 98.88% |
| Sodium chloride (10 mM) | 99.90% | 99.24% | 98.56% |
| Sodium chloride (50 mM) | 99.90% | 99.09% | 98.06% |
| Sodium sulphate (10 mM) | — | — | — |
| Sodium sulphate (50 mM) | — | — | — |

TABLE 3
Stability of insulin glargine (100 U/ml) assessed by RP-HPLC
following storage at 30° C. for 4 and 12 weeks. All formulations
contained 25 mM m-cresol, 185 mM glycerol and 30 μg/ml ionic
zinc and were adjusted to pH 4.0.

| | RP-HPLC | | |
|---|---|---|---|
| | main | RP-HPLC main | RP-HPLC main |
| | peak | peak | peak |
| Additive | (0 weeks) | (4 weeks) | (12 weeks) |
| None | 99.51% | 98.24% | 95.76% |
| Sodium chloride (10 mM) | 99.51% | 98.15% | 95.05% |
| Sodium chloride (50 mM) | 99.50% | 97.11% | 89.53% |
| Sodium sulphate (10 mM) | — | — | — |
| Sodium sulphate (50 mM) | — | — | — |

### Example 2—Effect of Triethylenetetramine (TETA) On the Stability of Insulin Glargine (100 U/ml)

The effect of TETA as an additive on the stability of insulin glargine (100 U/ml) was studied by comparing the stability of insulin glargine (100 U/ml) in the currently marketed formulation of Lantus® both in the absence and in the presence of TETA. The currently marketed formulation of Lantus® contains 25 mM m-cresol, 185 mM glycerol and 30 μg/ml ionic zinc and is adjusted to pH 4.0.

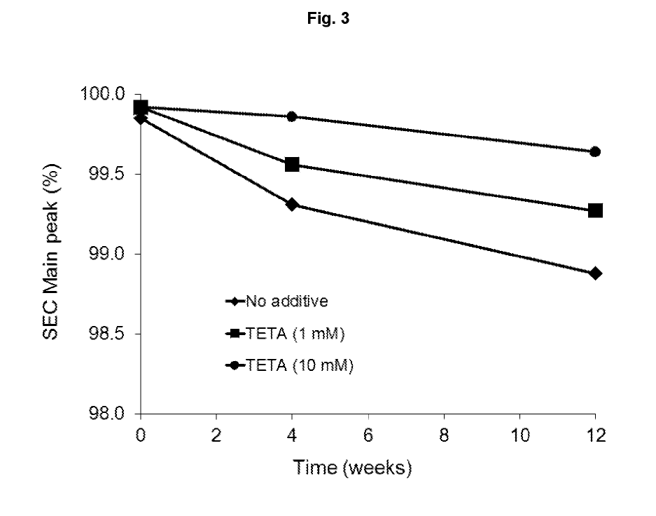

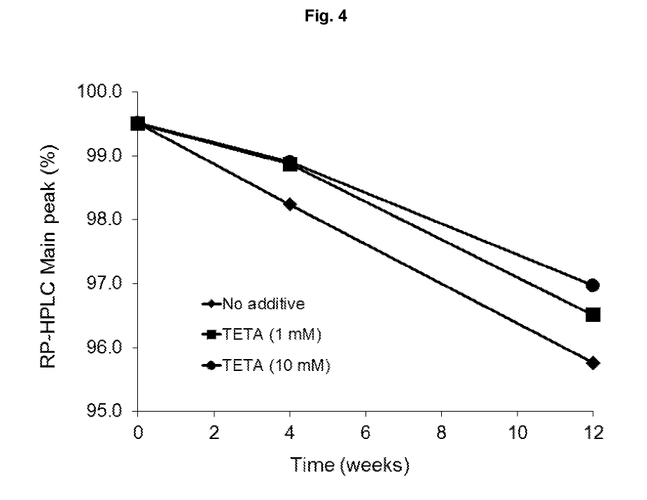

It can be seen from Tables 4-6 below (and FIGS. 3-4) that the addition of TETA resulted in improvement of both physical stability (assessed by visual assessment and SEC (see General Procedures)) and chemical stability (assessed by RP-HPLC (see General Procedures)) of insulin glargine in the formulation. The stability improvement observed was greater if 10 mM of TETA was used compared with using lower concentration of 1 mM of TETA.

TABLE 4
Stability of insulin glargine (100 U/ml) assessed by visual assessment
following storage at 30° C. for 4 and 12 weeks. All formulations
contained 25 mM m-cresol and 185 mM glycerol and were adjusted
to pH 4.0. Extent of visible precipitation is graded on a scale 1-3;
1 = clear solution free of visible particles; 2 = slight particle
formation, 3 = more significant precipitation.

| | Visual | Visual | Visual |
|---|---|---|---|
| | assessment | assessment | assessment |
| Additive | (0 weeks) | (4 weeks) | (12 weeks) |
| None | 1 | 1 | 2 |
| TETA (1 mM) | 1 | 1 | 1 |
| TETA (10 mM) | 1 | 1 | 1 |

TABLE 5
Stability of insulin glargine (100 U/ml) assessed by SEC following
storage at 30° C. for 4 and 12 weeks. All formulations
contained 25 mM m-cresol, 185 mM glycerol and 30 μg/ml
ionic zinc and were adjusted to pH 4.0.

| | SEC main | SEC main | SEC main |
|---|---|---|---|
| | peak | peak | peak |
| Additive | (0 weeks) | (4 weeks) | (12 weeks) |
| None | 99.85% | 99.31% | 98.88% |
| TETA (1 mM) | 99.92% | 99.56% | 99.27% |
| TETA (10 mM) | 99.92% | 99.86% | 99.64% |

TABLE 6
Stability of insulin glargine (100 U/ml) assessed by RP-HPLC
following storage at 30° C. for 4 and 12 weeks. All formulations
contained 25 mM m-cresol,185 mM glycerol and 30 μg/ml ionic
zinc and were adjusted to pH 4.0.

| | RP-HPLC | RP-HPLC | RP-HPLC |
|---|---|---|---|
| | main peak | main peak | main peak |
| Additive | (0 weeks) | (4 weeks) | (12 weeks) |
| None | 99.51% | 98.24% | 95.76% |
| TETA (1 mM) | 99.51% | 98.87% | 96.51% |
| TETA (10 mM) | 99.50% | 98.90% | 96.97% |

### Example 3—Effect of Triethylenetetramine (TETA) On Stability of Insulin Glargine (100 U/ml)—Further Study

An additional experiment was performed to study the effect of TETA as additive on the stability of insulin glargine (100 U/ml). The effect was studied by comparing the stability of insulin glargine (100 U/ml) in the currently marketed formulation of Lantus® both in the absence and in the presence of specific concentrations of TETA. The currently marketed formulation of Lantus® contains 25 mM m-cresol, 185 mM glycerol and 30 μg/ml ionic zinc and is adjusted to pH 4.0.

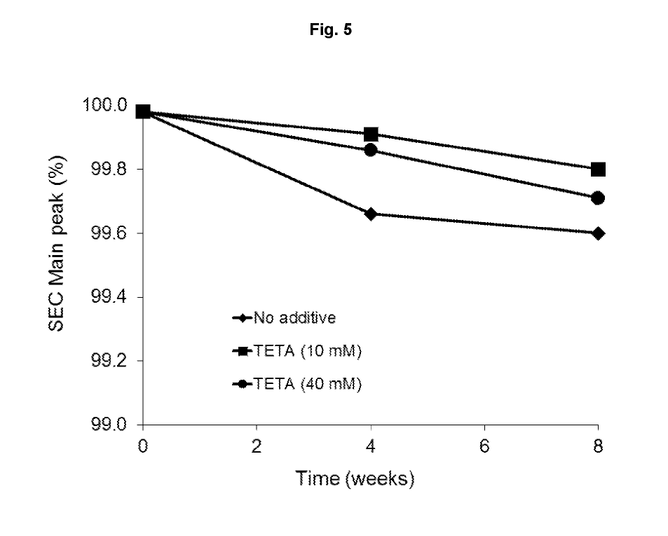

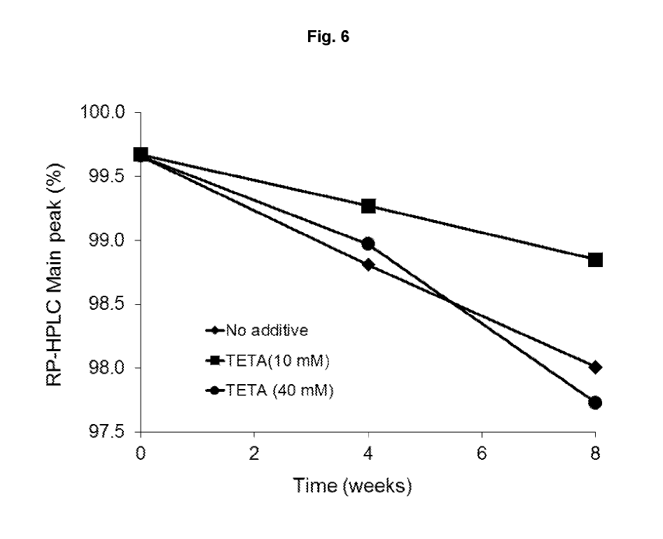

Confirming the findings of Example 2, an improvement in physical and chemical stability of insulin glargine was observed on addition of TETA, as can be seen from Tables 7-9 below (and FIGS. 5-6). The magnitude of the stability improvement was greatest if 10 mM of the additive was used compared with higher additive concentrations.

TABLE 7
Stability of insulin glargine (100 U/ml) assessed by visual assessment
following storage at 30° C. for 4 and 8 weeks. All formulations
contained 25 mM m-cresol, 185 mM glycerol and 30 μg/ml ionic
zinc and were adjusted to pH 4.0. Extent of visible precipitation is
graded on a scale 1-3; 1 = clear solution free of visible particles; 2 =
slight particle formation, 3 = more significant precipitation.

| | Visual | Visual | Visual |
|---|---|---|---|
| | assessment | assessment | assessment |
| Additive | (0 weeks) | (4 weeks) | (8 weeks) |
| None | 1 | 1 | 2 |
| TETA (10 mM) | 1 | 1 | 1 |
| TETA (40 mM) | 1 | 1 | 1 |

TABLE 8
Stability of insulin glargine (100 U/ml) assessed by SEC following
storage at 30° C. for 4 and 8 weeks. All formulations contained
25 mM m-cresol, 185 mM glycerol and 30 μg/ml ionic zinc
and were adjusted to pH 4.0.

| | SEC main | SEC main | SEC main |
|---|---|---|---|
| | peak | peak | peak |
| Additive | (0 weeks) | (4 weeks) | (8 weeks) |
| None | 99.98% | 99.66% | 99.60% |
| TETA (10 mM) | 99.98% | 99.91% | 99.80% |
| TETA (40 mM) | 99.98% | 99.86% | 99.71% |

TABLE 9
Stability of insulin glargine (100 U/ml) assessed by RP-HPLC
following storage at 30° C. for 4 and 8 weeks. All formulations
contained 25 mM m-cresol, 185 mM glycerol and 30 μg/ml ionic
zinc and were adjusted to pH 4.0.

| | RP-HPLC | RP-HPLC | RP-HPLC |
|---|---|---|---|
| | main peak | main peak | main peak |
| Additive | (0 weeks) | (4 weeks) | (8 weeks) |
| None | 99.66% | 98.81% | 98.01% |
| TETA (10 mM) | 99.67% | 99.27% | 98.85% |
| TETA (40 mM) | 99.66% | 98.97% | 97.73% |

### Example 4—Effect of Triethylenetetramine (TETA) On the Stability of Insulin Glargine (500 U/ml)

The effect of TETA on the stability of insulin glargine (500 U/ml) was studied by comparing the stability of insulin glargine (500 U/ml) formulated in the currently marketed formulation of Lantus® (Control formulation) both in the absence and in the presence of TETA. The zinc level in all compositions tested was adjusted to account for the increased concentration of insulin glargine so that the weight ratio between insulin glargine and zinc was the same as that in the marketed Lantus® formulation. The Control formulation thus contained 500 U/ml insulin glargine, 25 mM m-cresol, 185 mM glycerol and 150 pg/ml ionic zinc and was adjusted to pH 4.0.

Tables 10-12 below show that the addition of TETA resulted in improvement of both physical stability (assessed by visual assessment and SEC (see General Procedures)) and chemical stability (assessed by RP-HPLC (see General Procedures)) of insulin glargine. Replacing glycerol with trehalose as a tonicity modifier also led to further slight improvement of stability.

TABLE 10
Stability of insulin glargine (500 U/ml) assessed by visual assessment
following storage at 30° C. for 4 and 8 weeks. All formulations
contained 25 mM m-cresol and 150 μg/ml ionic zinc and were adjusted
to pH 4.0. Extent of visible precipitation is graded on a scale 1-3;
1 = clear solution free of visible particles; 2 = slight particle
formation, 3 = more significant precipitation.

| | Visual | Visual | Visual |
|---|---|---|---|
| | assessment | assessment | assessment |
| Additive | (0 weeks) | (4 weeks) | (8 weeks) |
| None | 1 | 2 | 3 |
| TETA (10 mM) + glycerol | 1 | 1 | 1 |
| (185 mM) | | | |
| TETA (10 mM) + trehalose | 1 | 1 | 1 |
| (185 mM) | | | |

TABLE 11
Stability of insulin glargine (500 U/ml) assessed by SEC following
storage at 30° C. for 4 and 8 weeks. All formulations
contained 25 mM m-cresol and 150 μg/ml ionic zinc and
were adjusted to pH 4.0.

| | SEC main | SEC main | SEC main |
|---|---|---|---|
| | peak | peak | peak |
| Additive | (0 weeks) | (4 weeks) | (8 weeks) |
| None | 99.99% | 99.87% | 99.80% |
| TETA (10 mM) + glycerol (185 mM) | 99.99% | 99.94% | 99.89% |
| TETA (10 mM) + trehalose | 99.99% | 99.96% | 99.92% |
| (185 mM) | | | |

TABLE 12
Stability of insulin glargine (500 U/ml) assessed by RP-HPLC
following storage at 30° C. for 4 and 8 weeks. All formulations
contained 25 mM m-cresol and 150 μg/ml ionic zinc
and were adjusted to pH 4.0.

| | RP-HPLC | RP-HPLC | RP-HPLC |
|---|---|---|---|
| | main peak | main peak | main peak |
| Additive | (0 weeks) | (4 weeks) | (8 weeks) |
| None | 99.76% | 99.34% | 98.84% |
| TETA (10 mM) + glycerol (185 mM) | 99.78% | 99.56% | 99.38% |
| TETA (10 mM) + trehalose | 99.76% | 99.65% | 99.57% |
| (185 mM) | | | |

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

SEQUENCE LISTING

SEQ ID NO. 1:
Chain A: NH2-Gly-Ile-Val-Glu-Gln-Cys-Cys-Thr-Ser-
Ile-Cys-Ser-Leu-Tyr-Gln-Leu-Glu-Asn-Tyr-Cys-Gly-
COOH
SEQ ID NO. 2:
Chain B: NH2-Phe-Val-Asn-Gln-His-Leu-Cys-Gly-Ser-
His-Leu-Val-Glu-Ala-Leu-Tyr-Leu-Val-Cys-Gly-Glu-
Arg-Gly-Phe-Phe-Tyr-Thr-Pro-Lys-Thr-Arg-Arg-COOH

1. An aqueous solution composition comprising insulin glargine as an active ingredient and triethylenetetramine (TETA) as a stabilising agent.

2. An aqueous solution composition according to claim 1, wherein the concentration of insulin glargine is between 10 U/ml and 1000 U/ml, for example between 50 U/ml and 500 U/ml, or between 100 U/ml and 200 U/ml.

3. An aqueous solution composition according to claim 1, wherein the concentration of insulin glargine is between 200 U/ml and 500 U/ml.

4. An aqueous solution composition according to claim 1, wherein the concentration of TETA is 1-50 mM, for example 2-45 mM, 5-40 mM or 2-25 mM such as 5-15 mM, such as about 10 mM.

5. An aqueous solution composition according to claim 1, wherein the pH is between 3 and 5, such as about pH 4.

6. An aqueous solution composition according to claim 1, further comprising a tonicity modifier.

7. An aqueous solution composition according to claim 6, wherein the tonicity modifier is an uncharged tonicity modifier and is selected from glycerol, 1,2-propanediol, mannitol, sorbitol, trehalose, PEG300 and PEG400.

8. An aqueous solution composition according to claim 1, further comprising a surfactant.

9. An aqueous solution composition according to claim 8, wherein the surfactant is a non-ionic surfactant.

10. An aqueous solution composition according to claim 8, wherein the surfactant is a cationic surfactant.

11. An aqueous solution composition according to claim 10, wherein the cationic surfactant is selected from a benzalkonium salt and a benzethonium salt.

12. An aqueous solution composition according to claim 11, wherein the cationic surfactant
is selected from benzethonium salts such as benzethonium chloride;
is selected from benzalkonium salts such as benzalkonium chloride; or
is a mixture of benzethonium salts and benzalkonium salts such as a mixture of benzethonium chloride and benzalkonium chloride.

13. (canceled)

14. (canceled)

15. An aqueous solution composition according to claim 1, which additionally comprises a preservative such as a phenolic or benzylic preservative.

16. An aqueous solution composition according to claim 15, wherein the phenolic or benzylic preservative is selected from the group consisting of phenol, m-cresol, chlorocresol, benzyl alcohol, propyl paraben and methyl paraben.

17. An aqueous solution composition according to claim 1, which is a therapeutic composition.

18. (canceled)

19. A method of treating diabetes mellitus comprising administering to a subject in need thereof an effective amount of an aqueous solution composition according to claim 1.

20. (canceled)

21. A method of improving the stability of an aqueous solution composition comprising insulin glargine as an active ingredient which comprises adding TETA to the composition as a stabilising agent.

22. A container containing one dose or a plurality of doses of an aqueous solution composition according to claim 1.

23. A container according to claim 22, which is a vial.

24. An injection device for single or multiple-use comprising a container according to claim 22 together with an injection needle.

25. An injection device according to claim 24, in the form of a pen.
